(19) United States
(12) Patent Application Publication
(10) Pub. No.: US 2024/0277816 A1
(43) Pub. Date: Aug. 22, 2024

(54) POLYPEPTIDE CONJUGATE AND APPLICATION THEREOF IN PREPARATION OF DRUG FOR TREATING DISEASES RELATED TO GLUCOSE METABOLISM

(71) Applicant: PegBio Co., Ltd., Jiangsu (CN)

(72) Inventors: Min XU, Jiangsu (CN); Yinghui ZHANG, Jiangsu (CN)

(73) Assignee: PegBio Co., Ltd., Jiangsu (CN)

(21) Appl. No.: 18/576,449

(22) PCT Filed: Jul. 20, 2022
(86) PCT No.: PCT/CN2022/106745

§ 371 Date: Jan. 4, 2024

(30) Foreign Application Priority Data
Jul. 20, 2021 (CN) 202110819329.0

(51) Int. Cl. A61K 38/26; A61K 47/60; A61P 3/08

(52) U.S. Cl. CPC A61K 38/26; A61K 47/60; A61P 3/08

(57) ABSTRACT

The present invention belongs to the field of medicine, and the present invention relates, in particular, to a polypeptide conjugate and an application of a polypeptide conjugate in the preparation of a drug for treating diseases related to glucose metabolism. The polypeptide conjugate of the present invention is formed by conjugating a glucagon mutant with one or more polyethylene glycols. The polypeptide conjugate can rapidly increase the blood glucose level within the body and maintain for a long time a stable blood glucose level within the body. The present invention is suitable for the treatment of congenital hyperinsulinism and various types of hypoglycemic disorders.

### PRIORITY AND RELATED APPLICATION

The present application is based upon and claims the benefit of a priority of Chinese Patent Application No. 202110819329.0 with a title of “Use of Polypeptide Conjugate in Preparation of Drug for Treating Diseases Related to Glucose Metabolism”, filed before the CNIPA on Jul. 20, 2021, the entire contents of which are incorporated herein by reference.

A sequence listing in electronic (XML file) format is filed with this application and incorporated herein by reference. The name of the XML file is “P70142_SL.xml”: the file was created on Mar. 28, 2024: the size of the file is 16,548 bytes.

### TECHNICAL FIELD

The present disclosure belongs to the field of medicine, and specifically, the present disclosure relates to a polypeptide conjugate and the use of the polypeptide conjugate in preparation of drug for treating diseases related to glucose metabolism.

### BACKGROUND

The inducing factors of hypoglycemia symptoms are complex, and hypoglycemia symptoms may be accompanied by many diseases. Congenital hyperinsulinism (CHI) is a disease leading to hypoglycemia caused by excessive secretion of insulin due to various congenital causes, which is the most common cause of severe and persistent hypoglycemia in neonate and child. According to statistics, the incidence of CHI among live-born infants is 1/30,000 to 1/50,000, and the incidence of CHI may be as high as 1/2,500 in countries and regions (such as Arabia) with relatively high intermarriage within a closed loop^{[1]}. Persistent and recurrent hypoglycemia and inappropriate insulin secretion under hypoglycemia are important clinical features of CHI. Because repeated hypoglycemia in CHI patients may lead to irreversible central nervous system damage or even death, early identification, diagnosis and reasonable treatment are very important.

Hypoglycemia may also be caused by excessive iatrogenic insulin and improper use of sulfonylureas, or accompanied by factors such as pregnancy, postoperative, tumor, liver diseases and other factors. When hypoglycemia occurs, the levels of glucose, ketone and lactose, which are mainly used by brain cells, cannot be maintained, and the energy supply from protein and fat in the body is blocked, resulting in damage to brain cells. Therefore, there is an urgent need in the art to develop timely and effective treatment methods for hypoglycemia-related diseases, which may maintain blood glucose levels for a long time.

The morphology of CHI is diverse, and the two most common histological types are focal and diffuse. Among them, the focal lesion is a proliferative adenoma, usually with a small lesion range. The abnormal pancreatic islet β cells in the center of the lesion have large nuclei, rich cytoplasm and irregular shape, and the abnormal pancreatic islet β cells are extremely active, which leads to an extremely high insulin secretion level in the whole lesion. Diffuse lesions affect all β cells of pancreatic islet, and pancreatic islet β cells generally have larger nuclei and more cytoplasm, and their insulin secretion function is vigorous. Genetically, the mutation of key genes involved in regulating insulin secretion is the key cause of CHI, and the mutation of genes such as ABCC8, KCNJ11, CLUD1, GCK, HADH, SLC16A1, HNF4A has been confirmed to be related to CHI, wherein diffuse CHI is most commonly caused by recessive mutation of K_{ATP} channel coding genes (ABCC8, KCNJ11), accounting for about 40-45%^{[2]}.

The therapeutic goal of CHI in infancy is to reduce the damage of hypoglycemia to the central nervous system. Surgery is an optional treatment. For focal CHI, the focus may be directly removed. For diffuse CHI, the situation is more complicated, and it is difficult to achieve complete cure by local resection. Most cases need subtotal pancreatectomy (resection of 95% to 98% pancreas), and this kind of surgery has a high risk of developing pancreatic exocrine dysfunction and diabetes, which requires lifelong pancreatic enzyme replacement and insulin treatment. Diabetes may occur immediately after surgery or may be found during postoperative follow-up^{[3]}. Meanwhile, in subtotal pancreatectomy, although 95% to 98% of pancreas is removed, some children may still show hyperinsulinemic hypoglycemia^{[4]}.

In terms of drug therapy, Diazoxide, a K_{ATP} channel opener, is a long-term first-line drug for the treatment of CHI abroad. Diazoxide has biological effects by binding with Sur1 subunit of K_{ATP} channel, so it requires the function of K_{ATP} channel to be complete, but a considerable number of patients with mutations in K_{ATP} channel coding genes (such as diffuse CHI with ABCC8 and KCNJ11 mutations) do not respond to diazoxide therapy. In addition, diazoxide has not been listed in China at present, and this drug is not available. Octreotide, a second-line drug, may be used to treat children with CHI who do not respond to diazoxide therapy. Octreotide is a synthetic long-acting somatostatin analogue, which inhibits insulin secretion by binding with somatostatin receptors 2 and 5 (SSTRs 2 and 5). The use of Octreotide in most children has a good initial therapeutic effect, but it often leads to rapid tolerance soon after administration, which limits its long-term application^{[5]}. In addition, Octreotide also has various side effects, including gastrointestinal reaction, gallbladder dilatation with gallstones and slow growth and development^{[6]}.

Human preproglucagon is a precursor polypeptide with 158 amino acids, which is differentially processed in tissues to form a variety of structurally related proglucagon-derived peptides, including glucagon (GCG), glucagon-like peptide-1(GLP-1), glucagon-like peptide-2(GLP-2) and oxyntomodulin (OXM). These molecules are involved in many physiological functions, including glucose homeostasis, insulin secretion, gastric emptying and intestinal growth, as well as food intake regulation. Among them, glucagon is a polypeptide containing 29 amino acids, corresponding to amino acids 53 to 81 of preproglucagon. Glucagon helps to maintain glucose levels in the blood by binding to glucagon receptors on liver cells and glycogen decomposition so that the liver releases glucose stored in the form of glycogen. With the depletion of these stores, glucagon also stimulates the liver to synthesize additional glucose through gluconeogenesis.

Due to its poor physical and chemical stability and short half-life, glucagon is mainly used in the first aid of hypoglycemia^{[7]}, and the duration of the drug action is short, and it is not suitable for long-term application. Dasiglucagon, a glucagon analogue developed by Zealand, is currently undergoing clinical trials for CHI. Although the drug partially overcomes the problem of physical and chemical stability, the action time of the drug is still short, and it is necessary to use a special perfusion pump for continuous infusion to children, which is inconvenient and costly.

### CITED DOCUMENTS

[1] Dillon PA, Curr Opin Pediatr, 2013, 25(3):357-361.
[2] Bellanne-Chantelot C, J Med Genet, 2010, 47(11):752-759.
[3] Arya VB, Plos One, 2014, 9: e98054.
[4] Beltrand J, Diabetes Care, 2012, 35:198-203.
[5] Demirbilek, J Clin Endocrinol, 2014, 99(10): 3660-3667.
[6] Levy-Khademi F, J Pediatr Endocrinol Metab, 2015, 28:449-451: Celik N, Horm Res Paediatr, 2013, 80:299-303.
[7] Hussain K, Horm Res, 2008, 69:2-13.

### SUMMARY

### Technical Problem

To solve the problems in the prior art, for example, glucagon drugs have poor stability, short action time, may only improve the blood glucose level of patients in a short period of time, and cannot be used for a long time, the present disclosure provides a polypeptide conjugate formed by conjugation of a glucagon mutant with a polyethylene glycol group, which may maintain the blood glucose level in mammals for a long time and meet the requirements of treatment for diseases such as congenital hyperinsulinism.

### Solution to Problem

In the first aspect, the present disclosure provides the use of a polypeptide conjugate formed by conjugation of a glucagon mutant with one or more polyethylene glycols in the preparation of a drug for treating a disease related to glucose metabolism:

wherein the glucagon mutant has substitution, addition or modification of amino acid at one or more positions of a sequence shown in SEQ ID NO: 1, so that a side chain of at least one amino acid contains a sulfhydryl group, and the polyethylene glycol is conjugated to the glucagon mutant through the sulfhydryl group.

In some embodiments, the use according to the present disclosure, wherein the substitution, addition or modification of amino acid includes at least one of the following (a) and (b):
(a) the amino acid at one or more positions of the sequence shown in SEQ ID NO: 1 being replaced with cysteine,
(b) one or more cysteines being added at one or more positions of the sequence shown in SEQ ID NO: 1.

In some embodiments, the use according to the present disclosure, wherein the substitution, addition or modification of amino acid is that a sulfhydryl-containing group is connected to the side chain of the amino acid at one or more positions of the sequence shown in SEQ ID NO: 1;
preferably, at least one of the following (c) to (g) is included:
(c) a sulfhydryl-containing group being connected to the side chain of glutamine at position 3,
(d) a sulfhydryl-containing group being connected to the side chain of lysine at position 12,
(e) a sulfhydryl-containing group being connected to the side chain of glutamine at position 20,
(f) a sulfhydryl-containing group being connected to the side chain of glutamine at position 24,
(g) a sulfhydryl-containing group being connected to the side chain of asparagine at position 28;
more preferably, a sulfhydryl-containing group is connected to the side chain of glutamine at position 24 of the sequence shown in SEQ ID NO: 1.

In some embodiments, the use according to the present disclosure, wherein the sulfhydryl-containing group is —C(═O)—Z₁—SH or —Z₁—SH, Z₁ is an unsubstituted or amino-substituted C₁₋₄ alkyl, the non-sulfhydryl side of the sulfhydryl-containing group is connected with the amino group in the side chain of the amino acid: the sulfhydryl-containing group is preferably —C(═O)—CH(NH₂)—CH₂—SH, —CH₂—SH, —CH₂—CH₂—SH or —CH₂—CH₂—CH₂—SH; and the sulfhydryl-containing group is more preferably —CH₂—CH₂—SH.

In some embodiments, the use according to the present disclosure, wherein the glucagon mutant has a sequence shown in any one of the following sequences:

(SEQ ID NO: 2)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln
(-CH₂—CH₂—SH)-Trp-Leu-Met-Asn-Thr-OH,
(SEQ ID NO: 3)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln
(-CH₂—CH₂—SH)-Trp-Leu-Met-Asn-Thr-NH₂,
(SEQ ID NO: 4)
H-His-Ser-Gln-Gly-Thr-Phe-Thr-Ser-Asp-Tyr-Ser-Lys
(-C(═O)—CH(NH₂)-CH₂—SH)-Tyr-Leu-Asp-Ser-Arg-Arg-
Ala-Gln-Asp-Phe-Val-Gln-Trp-Leu-Met-Asn-Thr-OH,
(SEQ ID NO: 5)
H-His-Ser-Gln-Gly-Thr-Phe-Thr-Ser-Asp-Tyr-Ser-Lys
(-C(═O)—CH(NH₂)-CH₂—SH)-Tyr-Leu-Asp-Ser-Arg-Arg-
Ala-Gln-Asp-Phe-Val-Gln-Trp-Leu-Met-Asn-Thr- NH₂,
(SEQ ID NO: 6)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Cys-
Trp-Leu-Met-Asn-Thr-OH,
(SEQ ID NO: 7)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Cys-
Trp-Leu-Met-Asn-Thr- NH₂,
(SEQ ID NO: 8)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Cys-OH,
(SEQ ID NO: 9)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Cys-NH₂.

In some embodiments, the use according to the present disclosure, wherein the polyethylene glycol has a molecular weight of 1 KDa to 60 KDa, more preferably 5 KDa to 50 KDa, further preferably 10 KDa to 40 KDa, further more preferably 15 KDa to 30 KDa, and most preferably 21 KDa to 29 KDa.

In some embodiments, the use according to the present disclosure, wherein the polypeptide conjugate has a structure shown in formula (I-1) or (I-1′):

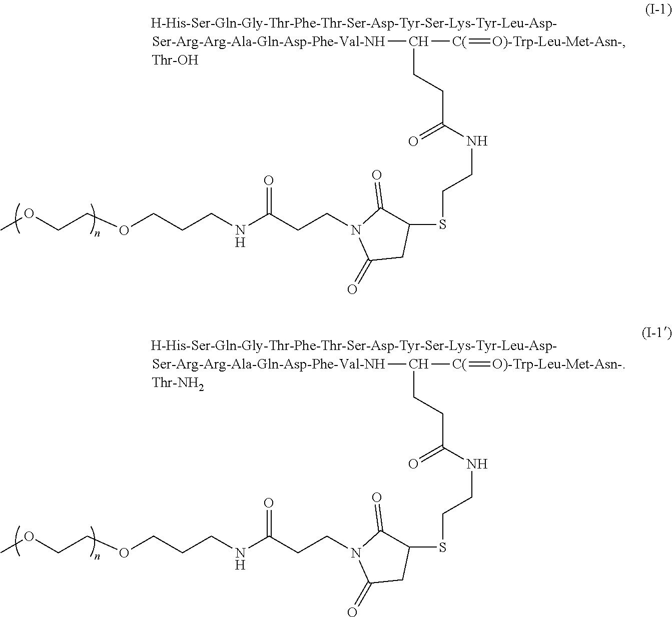

In some embodiments, the use according to the present disclosure, wherein the disease related to glucose metabolism is hypoglycemia.

In some embodiments, the use according to the present disclosure, wherein the hypoglycemia is any one selected from congenital hyperinsulinemic hypoglycemia, diabetic hypoglycemia, non-diabetic hypoglycemia, reactive hypoglycemia, fasting hypoglycemia, gestational hypoglycemia, drug-induced hypoglycemia, surgery-induced hypoglycemia and tumor-induced hypoglycemia.

In some embodiments, the use according to the present disclosure, wherein the disease related to glucose metabolism is congenital hyperinsulinism.

In the second aspect, the present disclosure provides the use of a polypeptide conjugate in the preparation of a drug for treating a subject suffering from a disease related to glucose metabolism, wherein the polypeptide conjugate and the specific types of the disease related to glucose metabolism is the same as defined in the first aspect of the present disclosure; preferably, the polypeptide conjugate or a composition comprising the polypeptide conjugate is administered to a subject in vivo: alternatively, the composition further comprises one or more pharmaceutically acceptable carriers.

In the third aspect, the present disclosure provides a polypeptide conjugate for treating a disease related to glucose metabolism, wherein the polypeptide conjugate is the same as defined in the first aspect of the present disclosure:
preferably, the polypeptide conjugate is used for treating a subject suffering from a disease related to glucose metabolism;
preferably, the disease related to glucose metabolism is congenital hyperinsulinism;
alternatively, the disease related to glucose metabolism is hypoglycemia, which is any one selected from congenital hyperinsulinemic hypoglycemia, diabetic hypoglycemia, non-diabetic hypoglycemia, reactive hypoglycemia, fasting hypoglycemia, gestational hypoglycemia, drug-induced hypoglycemia, surgery-induced hypoglycemia and tumor-induced hypoglycemia.

In the fourth aspect, the present disclosure provides a method for treating a disease related to glucose metabolism, wherein the method comprises administering to a subject a therapeutically effective amount of the polypeptide conjugate as defined in the first aspect of the present disclosure:
preferably, the disease related to glucose metabolism is congenital hyperinsulinism;
alternatively, the disease related to glucose metabolism is hypoglycemia, which is any one selected from congenital hyperinsulinemic hypoglycemia, diabetic hypoglycemia, non-diabetic hypoglycemia, reactive hypoglycemia, fasting hypoglycemia, gestational hypoglycemia, drug-induced hypoglycemia, surgery-induced hypoglycemia and tumor-induced hypoglycemia.

### Effects

In the polypeptide conjugate provided by the present disclosure, although being modified by PEGylation, glucagon mutant polypeptide may still maintain a high biological activity of glucagon. The polypeptide conjugate may rapidly raise blood glucose in a short time, and may play a long-term role in stabilizing blood glucose in different mammalian hypoglycemia models. The polypeptide conjugate may be applied to the treatment of congenital hyperinsulinism and various hypoglycemia diseases.

In some embodiments, the polypeptide conjugate shown in formula I-1 provided by the present disclosure has high glucagon activity, may raise blood glucose to a safe value in a short time, and maintain the long-term stability of blood glucose level in a hypoglycemia animal model, and has a long drug action time, so it is suitable for the treatment of diseases related to hypoglycemia induced by multiple factors, especially for the treatment of congenital hyperinsulinism.

### BRIEF DESCRIPTION OF THE DRAWINGS

FIG. 1 shows the changes of blood glucose in mice after injection of polypeptide conjugate I-1 and its precursor glucagon mutant Q-1.

FIG. 2 shows the glucose-raising effect of polypeptide conjugate I-1 on glimepiride hypoglycemia mouse model.

FIG. 3 shows the glucose-raising effect of polypeptide conjugate I-1 on hyperinsulinemic hypoglycemia rat model.

### DETAILED DESCRIPTION

### Definitions

Unless stated otherwise, the terms used in the present disclosure have the following meanings.

In the claims and/or specification of the present disclosure, the words “a” or “an” or “the” may refer to “one”, and may also refer to “one or more”, “at least one” and “one or more than one”.

As used in the claims and the specification, the words “comprising”, “having”, “including” or “containing” refer to inclusion or openness, not excluding additional, unquoted components or method steps. Meanwhile, “comprising”, “having”, “including” or “containing” may also mean closed, excluding additional, unquoted components or method steps.

Throughout the present application, the term “about” means that a value includes the standard deviation of the error caused by the device or method used to measure the value. The numerical ranges and parameters used to define the present disclosure are all approximate values, and the relevant numerical values in the specific examples have been presented as accurately as possible here. However, any numerical value inevitably contains the aforementioned standard deviation caused by the test method or device. Therefore, unless explicitly stated otherwise, it should be understood that all ranges, quantities, values and percentages used in the present disclosure have been modified by the word “about”. Here, “about” typically means that the actual value is within plus or minus 10%, 5%, 1% or 0.5% of a specific value or range.

The terms “polypeptide” and “protein” may interchangeably refer to a string of at least two amino acid residues connected to each other via covalent bonds (such as peptide bonds), which may be recombinant polypeptide, natural polypeptide or synthetic polypeptide. A polypeptide may be linear or branched, it may contain modified amino acids and may be interrupted by non-amino acids. The term also includes amino acid polymers that have been modified (e.g., disulfide bond formation, glycosylation, lipidation, acetylation, phosphorylation or any other operation, such as conjugation with labeled components).

The term “amino acid” includes natural amino acids, unnatural amino acids, amino acid analogues and all D and L stereoisomers thereof.

The amino acids, abbreviations and English short names thereof in the present disclosure are as follows:

Histidine (His, H); Serine (Ser, S); Glutamine (Gln, Q); Gly (glycine, G); Threonine (Thr, T); Phenylalanine (Phe, F); Aspartic acid (Asp, D); Tyrosine (Tyr, Y); Leucine (Leu, L); Arginine (Arg, R); Alanine (Ala, A); Glutamine (Gln, Q); Valine (Val, V); Tryptophan (Trp, W); Leucine (Leu, L); Methionine (Met, M); Asparagine (Asn, N); Cysteine (Cys, C); Lysine (Lys, K).

The terms “mutant”, “mutant of a polypeptide”, “polypeptide mutant” and “polypeptide variant” refer to a polypeptide that contains changes (i.e., substitution, insertion, deletion and/or modification) at one or more (e.g., several) positions relative to a “wild-type” or “comparative” polypeptide. The mutant may be a fully functional mutant, a functional mutant lacking one or more activities, or a functional mutant having one or more enhanced activities. In some embodiments, the mutant of a polypeptide is a glucagon mutant.

Unless otherwise specified or marked, in the present disclosure, the N-terminal and C-terminal of the “polypeptide”, “mutant” or synonyms thereof may be independently in a modified or unmodified state; wherein the N-terminal may be naked (H—) or chemically modified, and the modifying groups include but are not limited to C₁₋₄ alkyl, acetyl, formyl, benzoyl, trifluoroacetyl, etc.; the C-terminal may be naked (—OH) or chemically modified, and the modification includes but is not limited to amidation (—NH₂).

In some embodiments, a “mutant of a polypeptide” comprises one or more amino acids that are substituted, added and/or modified. Substitution refers to replacing an amino acid occupying a position with a different amino acid. Deletion refers to the removal of an amino acid occupying a certain position. Addition refers to the addition of an amino acid adjacent to and immediately after an amino acid occupying a position. Modification may include modification of natural sequences, such as modification of functional groups, intramolecular covalent bonding (for example, cyclization between side chains), methylation, acylation, ubiquitination, phosphorylation, aminohexanation, biotinylation, etc., and it may also include substitution to form an unnatural compound, and/or addition of one or more amino acids to the carboxyl end and/or amino end of amino acids in the natural sequences.

The term “polypeptide conjugate” refers to a product obtained by covalently or noncovalently connecting one or more amino acids in a polypeptide or a mutant of a polypeptide with a modifying group. Modification methods include but are not limited to PEGylation, phosphorylation, methylation, glycosylation, biotinylation, acylation and so on. In some embodiments, the modifying group is a polyethylene glycol group.

The term “independently” means that at least two groups (or ring systems) within the same or similar value ranges in a structure may have the same or different meanings in a specific situation. For example, substituent X and substituent Y are independently hydrogen, hydroxyl, alkyl or aryl, then when substituent X is hydrogen, substituent Y may be hydrogen, hydroxyl, alkyl or aryl: similarly, when the substituent Y is hydrogen, the substituent X may be hydrogen, hydroxyl, alkyl or aryl.

The term “alkyl” includes linear, branched or cyclic saturated alkyl. For example, alkyl includes but is not limited to methyl, ethyl, propyl, cyclopropyl, n-butyl, isobutyl, sec-butyl, tert-butyl, cyclobutyl, n-pentyl, cyclohexyl and the like. Illustratively, “C₁₋₆” in “C₁₋₆ alkyl” refers to a group containing 1, 2, 3, 4, 5 or 6 carbon atoms arranged in a linear, branched or cyclic form.

The term “aryl” herein refers to an unsubstituted or substituted monocyclic or polycyclic aromatic group having carbon atoms, including 6- to 10-membered monocyclic or fused polycyclic carbon-ring group (i.e., rings sharing adjacent pairs of carbon atoms). For example, heteroaryl includes but is not limited to phenyl, 1-naphthyl, 2-naphthyl and the like.

The term “treatment” used in the context of the present disclosure means exposing (e.g., administering), to a subject after suffering from a disease, the compound of the present disclosure or a pharmaceutically acceptable salt, ester, solvate, optical isomer, tautomer, isotope label, prodrug thereof or a pharmaceutical composition containing the same (hereinafter also referred to as “pharmaceutical composition of the present disclosure”), so that the symptoms of the disease are alleviated as compared with those without exposure, which does not mean that it is necessary to completely inhibit the symptoms of the disease. Suffering from a disease means that the body has symptoms of the disease.

The terms “individual”, “patient” or “subject” used in the context of the present disclosure include mammals. Mammals include, but are not limited to, domestic animals (e.g., cattle, sheep, cats, dogs and horses), primates (e.g., humans and non-human primates such as monkeys), rabbits, and rodents (e.g., mice and rats).

The term “therapeutically effective amount” used in the context of the present disclosure refers to an amount that effectively achieves a desired therapeutic result at the required dosage and for the required period of time. The therapeutically effective amount of the compound or pharmaceutical composition of the present disclosure may vary according to many factors such as the disease state, the age, sex and weight of the individual, and the ability of the immune adjuvant or pharmaceutical composition to stimulate the desired response in the individual.

The term “pharmaceutical composition” used in the context of the present disclosure refers to a composition that may be used for medicine, which comprises a polypeptide conjugate or a pharmaceutically acceptable form thereof (e.g., salt, hydrate, solvate, stereoisomer, tautomer, metabolite, prodrug, etc.) and other components (e.g., pharmaceutically acceptable excipients).

The term “pharmaceutically acceptable excipient” or “pharmaceutically acceptable carrier” used in the context of the present disclosure refers to auxiliary materials widely used in the field of pharmaceutical production. The main purpose of using excipients is to provide a pharmaceutical composition having safe use, stable properties and/or specific functions, and also to provide a method so that after the drug is administered to a subject, the active ingredient may be dissolved at a desired rate, or that the active ingredient may be effectively absorbed in the administered subject. The pharmaceutically acceptable excipient may be an inert filler or a functional ingredient that provides a certain function (such as stabilizing the overall pH value of the composition or preventing the degradation of active ingredients in the composition) for the pharmaceutical composition. Non-limiting examples of the pharmaceutically acceptable excipient include, but are not limited to, binders, suspending agents, emulsifiers, diluents (or fillers), granulators, adhesives, disintegrants, lubricants, anti-adhesion agents, glidants, wetting agents, gelling agents, absorption retarders, dissolution inhibitors, enhancers, adsorbents, buffers, chelating agents, preservatives, colorants, correctants, sweeteners and the like.

The pharmaceutical composition of the present disclosure may be prepared by any method known to persons skilled in the art, for example, conventional mixing, dissolving, granulating, emulsifying, grinding, encapsulating, embedding and/or lyophilizing processes.

The term “hypoglycemia” refers to a healthy state in which the blood glucose level is lower than that of normal people, which is often caused when patients eat less than usual or do more activities or exercises than usual after taking oral hypoglycemic agents or insulin. In addition, hypoglycemia may be caused by drinking alcohol, taking drugs reducing glucose level, serious physical diseases, lack of hormones such as adrenocortical hormone and glucagon, tumors in insulin-producing pancreas, insulin autoimmune diseases, patients undergoing gastrectomy, congenital errors of carbohydrate metabolism disorders, etc. The specific hypoglycemia types may be congenital hyperinsulinemic hypoglycemia, diabetic hypoglycemia, non-diabetic hypoglycemia, reactive hypoglycemia, fasting hypoglycemia, pregnancy hypoglycemia, drug-induced hypoglycemia, surgery-induced hypoglycemia or tumor-induced hypoglycemia, which may be, but not have to be, separate or mutually exclusive.

The technical solution of the present disclosure will be described below in combination with specific examples. The following examples are provided to further illustrate the present disclosure, but not to limit the scope of the present disclosure. It is obvious for persons skilled in the art to make various changes and improvements for the specific implementation of the present disclosure without departing from the spirit and scope of the present disclosure.

The preparation of the compound of the present disclosure may be realized by synthetic methods well known to persons skilled in the art, including but not limited to the specific embodiments listed below, the embodiments formed by combining the foregoing with other chemical synthesis methods, and equivalent alternatives well known to persons skilled in the art. Preferable embodiments include but are not limited to the examples of the present disclosure. Known starting materials used in the present disclosure may be synthesized by methods known in the art or purchased by conventional commercial means.

### Polypeptide Conjugate

The polypeptide conjugate of the present disclosure is formed by conjugation of glucagon mutant with one or more polyethylene glycols; wherein the glucagon mutant has substitution, addition or modification of amino acid at one or more positions of the sequence shown in SEQ ID NO: 1, so that the side chain of at least one amino acid contains a sulfhydryl group, and the polyethylene glycol is conjugated to the mutant through the sulfhydryl group.

(SEQ ID NO: 1)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-OH

As generally known by those skilled in the art, in a bioactive molecule (for example, a bioactive polypeptide) conjugated with a polymer group, along with the increase of molecular weight of the polymer group, the biological activity of the conjugate molecule will decrease^{[8]}, and this conclusion is confirmed again by the in vitro test results of PEG conjugate of glucagon variant (see Chinese patent application CN201511017796.2). However, it is unexpectedly found by the present disclosure that after conjugated with polyethylene glycol, the glucagon mutant shows improved in vivo activity, and the polypeptide conjugate shows better rate and peak value of raising blood glucose than those of the unconjugated mutant, and has a more obvious glucose-raising effect. The strong in vivo activity allows the polypeptide conjugate to resist the antagonistic effect of high concentration insulin and have the potential to treat congenital hyperinsulinism. Moreover, the polypeptide conjugate may also achieve a long-term effect of stabilizing blood glucose, and has a longer drug action time compared with glucagon and polypeptide variants of glucagon, which can fully meet the clinical needs of patients with congenital hyperinsulinism and various types of hypoglycemia.

### <Glucagon Mutant>

Compared with the wild-type glucagon having the sequence shown in SEQ ID NO: 1, the glucagon mutant of the present disclosure has mutated amino acids at one or more positions in the sequence shown in SEQ ID NO: 1. The mutation modes of amino acids include but are not limited to substitution, addition or modification. In some embodiments, the mutation mode of amino acids is selected from one of substitution, addition and modification: in other embodiments, the mutation mode of amino acids is selected from the combination of two or more of substitution, addition and modification.

Further, the glucagon mutant has mutated amino acids at 1, 2, 3, 4, 5 or more positions in the sequence shown in SEQ ID NO: 1. Moreover, each mutated amino acid may be independently located at the N-terminal, C-terminal or inside the sequence shown in SEQ ID NO: 1, and the mutation mode of the amino acid at each position may be independently selected from one of substitution, addition and modification.

Through the substitution, addition or modification of amino acids at one or more positions, one or more amino acids in the glucagon mutant contain sulfhydryl groups in the side chain, providing a conjugation site for the conjugation with polyethylene glycol.

In the present disclosure, the substitution of amino acids includes replacing amino acids at one or more positions of the sequence shown in SEQ ID NO: 1 with cysteine. In some embodiments, the substitution of amino acids includes the substitution of cysteine for lysine at position 12 and cysteine for glutamine at position 24. In other embodiments, the substitution of amino acids is to replace the amino acid at any other position with cysteine.

In the present disclosure, the addition of amino acids includes the addition of one or more cysteines at one or more positions in the sequence shown in SEQ ID NO: 1. In some embodiments, the addition of amino acids includes the addition of 1, 2, 3, 4 or more cysteines at the C-terminus of the sequence shown in SEQ ID NO: 1. In other embodiments, the addition of amino acids further includes the addition of one or more cysteines at the N-terminus of the sequence shown in SEQ ID NO: 1, or at any position between the N-terminus and the C-terminus.

In the present disclosure, the modification of amino acids includes connecting sulfhydryl-containing groups to the side chains of amino acids at one or more positions in the sequence shown in SEQ ID NO: 1. Illustratively, the modification of amino acids includes the modification of one or more amino acids having an amino group in side chain, and the modification method includes connecting the amino group in the amino acid side chain to the non-sulfhydryl end of a sulfhydryl-containing group.

In some embodiments, the modification of amino acids includes at least one of the following (c)-(g): (c) a sulfhydryl-containing group is connected to the side chain of glutamine at position 3, (d) a sulfhydryl-containing group is connected to the side chain of lysine at position 12, (e) a sulfhydryl-containing group is connected to the side chain of glutamine at position 20, (f) a sulfhydryl-containing group is connected to the side chain of glutamine at position 24, and (g) a sulfhydryl-containing group is connected to the side chain of asparagine at position 28. In some preferable embodiments, the amino acid is modified by connecting a sulfhydryl-containing group to the side chain of glutamine at position 24.

In some more specific embodiments, the sulfhydryl-containing group is —C(═O)—Z₁—SH or —Z₁—SH, wherein Z₁ is an unsubstituted or amino-substituted C₁₋₄ alkyl, and the non-sulfhydryl side of the group is connected with the amino group in the side chain of the amino acid. Illustratively, Z₁ is methyl, ethyl, propyl, butyl, amino-substituted ethyl and the like.

In some preferable embodiments, the sulfhydryl-containing group is —C(═O)—CH(NH₂)—CH₂—SH, —CH₂—SH, —CH₂—CH₂—SH or —CH₂—CH₂—CH₂—SH. In some more preferable embodiments, the sulfhydryl-containing group is —CH₂—CH₂—SH.

In some embodiments, the “mutated amino acid” may also include added, deleted or replaced amino acids at one or several positions of the sequence shown in SEQ ID NO: 1 that do not affect the activity of the protein. As well known, the biological activity will not be basically changed if a few amino acid residues in certain regions of a polypeptide, such as unimportant regions, are changed. For example, a sequence obtained by proper substitution, addition or deletion of certain amino acids will not affect its activity.

In the present disclosure, the C-terminal and N-terminal of the glucagon mutant may be independently modified or unmodified groups. Illustratively, the modifying group includes, but is not limited to, amino, C₁₋₄ alkyl, acetyl, formyl, benzoyl, trifluoroacetyl, β-hydroxyimidazolyl, 4-imidazolyl, and β-carboxyimidazolyl. In some embodiments, the C-terminal group of the glucagon mutant is —OH and the N-terminal group is —H. In some embodiments, the C-terminal group of the glucagon mutant is —NH₂, and the N-terminal group is —H.

In some preferable embodiments, the glucagon mutant has the sequence shown in any one of SEQ ID NO: 2 to 9, and the specific sequences shown in SEQ ID NO: 2 to 9 are as follows:

(SEQ ID NO: 2)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln
(-CH₂—CH₂—SH)-Trp-Leu-Met-Asn-Thr-OH,
(SEQ ID NO: 3)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln
(-CH₂—CH₂—SH)-Trp-Leu-Met-Asn-Thr-NH₂,
(SEQ ID NO: 4)
H-His-Ser-Gln-Gly-Thr-Phe-Thr-Ser-Asp-Tyr-Ser-Lys
(-C(═O)—CH(NH₂)-CH₂—SH)-Tyr-Leu-Asp-Ser-Arg-Arg-
Ala-Gln-Asp-Phe-Val-Gln-Trp-Leu-Met-Asn-Thr-OH,
(SEQ ID NO: 5)
H-His-Ser-Gln-Gly-Thr-Phe-Thr-Ser-Asp-Tyr-Ser-Lys
(-C(═O)—CH(NH₂)-CH₂—SH)-Tyr-Leu-Asp-Ser-Arg-Arg-
Ala-Gln-Asp-Phe-Val-Gln-Trp-Leu-Met-Asn-Thr- NH₂,
(SEQ ID NO: 6)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Cys-
Trp-Leu-Met-Asn-Thr-OH,
(SEQ ID NO: 7)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Cys-
Trp-Leu-Met-Asn-Thr- NH₂,
(SEQ ID NO: 8)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Cys-OH,
(SEQ ID NO: 9)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Cys-NH₂.

### <Polyethylene Glycol>

In the present disclosure, polyethylene glycol (PEG) is conjugated with glucagon mutant through a thioether bond to obtain a polypeptide conjugate having significantly enhanced drug activity in vivo, thus achieving an effect of long-term stabilizing blood glucose. Polyethylene glycol comprises a highly reactive activating group for conjugation with a glucagon mutant. In some embodiments, polyethylene glycol contains a maleimide group, and the connection between an alkenyl group of the maleimide group and sulfhydryl group of glucagon mutant forms a thioether bond, resulting in a polypeptide conjugate of one or more polyethylene glycols conjugated with the glucagon mutant. In other embodiments, polyethylene glycol may also comprise halogen, vinyl sulfone, disulfide bond, sulfhydryl, aldehyde group, carbonyl group, O-substituted hydroxylamine, active ester, alkenyl, alkynyl, azide group or other activating groups having high chemical reactivity, and a thioether bond is formed by the reaction between the activating group and sulfhydryl group of the glucagon mutant, to obtain a polypeptide conjugate.

In the present disclosure, polyethylene glycol (PEG) does not have a particularly limited structure, and PEG molecules may be linear, branched or crotched. PEG includes both types in which the terminal group is hydroxyl and types in which the terminal group is other groups. Illustratively, other terminal groups include, but are not limited to, alkoxy, cycloalkyloxy, cycloalkylalkyloxy, alkenyl, aryloxy or aralkyloxy. These PEG molecular types are known in the prior art and are conventionally used in polypeptide modification.

In the present disclosure, polyethylene glycol (PEG) may have any required length of polymer chains. Because the polymer is composed of molecules having different polymerization degrees in a certain distribution range, the molecular weight of the polymer is typically represented by average molecular weight. Specifically, it may be a number average molecular weight or a weight average molecular weight. When the polymerization degree of polymers is quite different, the number average molecular weight and weight average molecular weight may have some deviation, but they tend to be equal for polymers with a narrow distribution range. As for the polyethylene glycol of the present disclosure, when the molecular weight is mentioned, it may be either a weight average molecular weight or a number average molecular weight, and the number average molecular weight is preferable. It should be noted that when the polypeptide conjugate comprises multiple polyethylene glycol molecules, the sum of the molecular weights of multiple polyethylene glycols is calculated.

In some embodiments, the molecular weight of polyethylene glycol is 1-60 KDa, more preferably 5-50 KDa, further preferably 10-40 KDa, further more preferably 15-30 KDa, and most preferably 21-29 KDa.

In some specific embodiments, the molecular weight of polyethylene glycol may be 15, 16, 17, 18, 19, 20, 21, 22, 23, 24, 25, 26, 27, 28, 29, 30, 31, 32, 33, 34, 35, 36, 37, 38, 39 and 40 KDa, and any numerical range between them.

In some optional embodiments, polyethylene glycol has a structure of

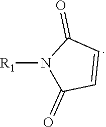

In this formula, R₁ may be selected from any one of the following structures:

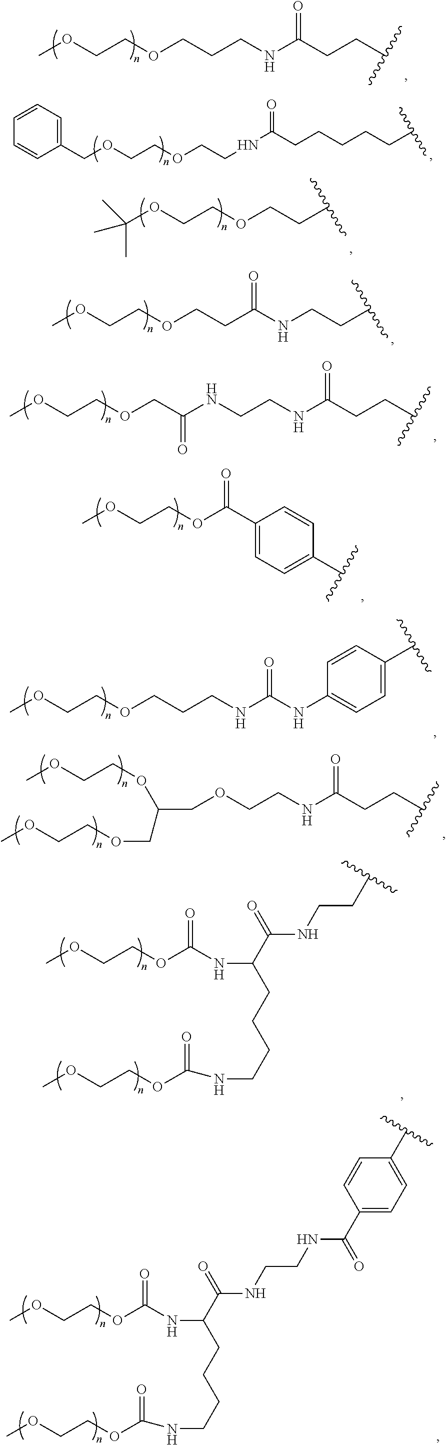

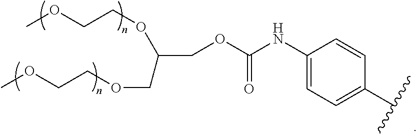

In some preferable embodiments, R₁ has the following structure:

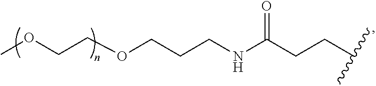

wherein each n is independently selected from any natural number, so that the molecular weight of polyethylene glycol falls within a target range.

In some preferable embodiments, the polypeptide conjugate has a structure shown in formula (I-1) or (I-1′):

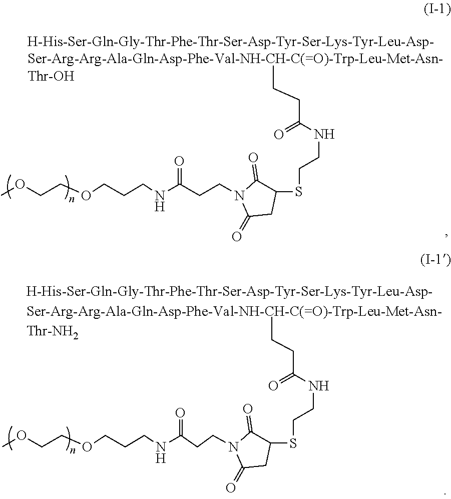

### Composition

The composition of the present disclosure comprises the polypeptide conjugate provided by the present disclosure, and the composition shows obviously enhanced biological activity of glucagon in vivo, is capable of effectively raising blood glucose and exerting a long-term drug effect of stabilizing blood glucose, and is suitable for long-term treatment of various diseases related to hypoglycemia symptoms.

In some embodiments, the composition further comprises one or more pharmaceutically acceptable carriers. In the present disclosure, the composition is used to promote the administration to organisms, facilitate the absorption of active ingredients to further exert biological activity. The composition of the present disclosure may be administered in any form, including injection (intra-arterial, intravenous, intramuscular, intraperitoneal, subcutaneous), mucosal, oral (oral solid preparation, oral liquid preparation), rectal, inhalation, implantation, topical (such as eye) administration, etc. Non-limiting examples of oral solid preparations include, but are not limited to, powder, capsule, troch, granule, tablet etc. Non-limiting examples of liquid preparations for oral or mucosal administration include, but are not limited to, suspension, tincture, elixir, solution etc. Non-limiting examples of preparations for topical administration include, but are not limited to, emulsion, gel, ointment, cream, patch, paste, foam, lotion, drops or serum preparation. Non-limiting examples of preparations for parenteral administration include, but are not limited to, solution for injection, dry powder for injection, suspension for injection, emulsion for injection etc. The composition of the present disclosure may also be made into controlled release formulations or delayed release formulations (such as liposomes or microspheres).

In the present disclosure, the route of administration may be changed or adjusted in any suitable way to meet the needs of the nature of drugs, the convenience of patients and medical staff and other related factors.

### Medical Use of Polypeptide Conjugate or Composition

The polypeptide conjugate of the present disclosure or the composition comprising the polypeptide conjugate is used for treating diseases related to glucose metabolism or preparing drugs for treating diseases related to glucose metabolism.

In some embodiments, the disease related to glucose metabolism is hypoglycemia. Specifically, diseases related to glucose metabolism are all kinds of diseases with hypoglycemia symptoms.

Induced by various factors, the body shows hypoglycemia symptoms. The polypeptide conjugate of the present disclosure or the composition comprising the polypeptide conjugate has an in vivo drug activity of improving the blood glucose level and stabilizing the blood glucose for a long time, so it is suitable for treating various diseases with hypoglycemia symptoms. Illustratively, hypoglycemia is selected from any one of the following: congenital hyperinsulinemic hypoglycemia, diabetic hypoglycemia, non-diabetic hypoglycemia, reactive hypoglycemia, fasting hypoglycemia, gestational hypoglycemia, drug-induced hypoglycemia, surgery-induced hypoglycemia and tumor-induced hypoglycemia.

In some embodiments, the disease related to glucose metabolism is congenital hyperinsulinism. Affected by factors such as genetic factors, congenital hyperinsulinism lacks long-term effective therapeutic drugs, which seriously affects the healthy development of infants and even threatens their lives. The polypeptide conjugate of the present disclosure or the composition comprising the polypeptide conjugate is capable of stabilizing the blood glucose level of the subject for a long time, and providing a therapeutic drug having great clinical application potential for the treatment of congenital hyperinsulinism.

In some embodiments, a polypeptide conjugate is used in the preparation of drugs for treating subjects suffering from diseases related to glucose metabolism. In some embodiment, a polypeptide conjugate or a composition comprising a polypeptide conjugate is suitable for use in a subject. Affected by the complex physiological environment in vivo, many drugs showing glucagon activity at the cellular level have no obvious drug activity in vivo and fail to play the role of glucagon for a long time. It is unexpectedly found by the present disclosure that, after the glucagon mutant is conjugated with polyethylene glycol, the polypeptide conjugate of the present disclosure shows significantly enhanced in-vivo drug activity and long-term drug action time in a subject, and it shows improved rate and peak value of raising blood glucose, is capable of stabilizing the blood glucose level in a subject for a long time. The polypeptide conjugate of the present disclosure is especially suitable for subjects suffering from diseases related to glucose metabolism and may play a long-term clinical therapeutic effect of stabilizing blood glucose.

### Examples

The other objects, characteristics and advantages of the present disclosure will become apparent from the following detailed description. However, it should be understood that the detailed description and specific examples (although indicating specific embodiments of the present disclosure) are given for explanatory purposes only, because various changes and modifications within the spirit and scope of the present disclosure will become obvious to those skilled in the art after reading the detailed description.

The reagents and raw materials used in the examples of the present disclosure are all commercially available conventional products unless otherwise specified.

### Example 1: Solid-Phase Synthesis of Glucagon Mutant

The glucagon mutant had the following structure:

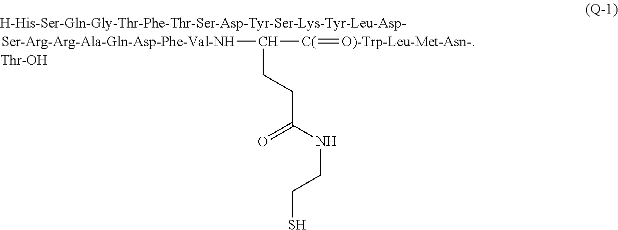

The synthesis steps of glucagon mutant Q-1 were as follows:

### Step 1: Swelling of Fmoc-Thr(tBu)-CTC Resin

Fmoc-Thr(tBu)-CTC resin was soaked in DMF for 2-5 hours to swell. After swelling, the resin was washed several times with DMF.

### Step 2: Deprotection of Fmoc

The Fmoc protecting group of Fmoc-Thr(tBu)-CTC resin was removed with a solution containing 20%(V/V) piperidine. After deprotection, DMF was used to wash the resin and then drained. The resin was sampled and tested with ninhydrin, and the test result should be positive.

### Step 3: Coupling Amino Acids

Amino acids No. 28 to No. 1 were coupled in sequence according to the amino acid sequence of a target mutant:

### 1) For Amino Acid No. 28 (Fmoc-L-Asn(Trt)-OH)

3.0 eq of Fmoc-L-Asn(Trt)-OH, 2.85 eq of TDBTU and DMF solvent were added to the deprotected resin. Then, 6.0 eq of DIPEA was added, to react at 20-30° C. for 60-120 minutes. The coupling reaction was monitored by ninhydrin test, and the test result should be negative. After the coupling was completed, the Fmoc protective agent was removed by the method in step 2, and ninhydrin was used for sampling and testing.

### 2) For Amino Acids No. 2-23, and 25-27

A DMF solution comprising 3 eq of Fmoc amino acid and 3 eq of HOBt was prepared, followed by ice bath, and then 3 eq of DIC was added to react for 10-15 minutes. Subsequently, the activated amino acid solution was added to the deprotected resin to react for 60-120 minutes, and the coupling reaction was monitored by ninhydrin test, and the test result should be negative. After the coupling was completed, the Fmoc protective agent was removed by the method in step 2, and ninhydrin was used for sampling and testing.

3) For Amino Acid No. 24 (Fmoc-L-Glu(NHCH₂CH₂STrt)-OH)

A DMF solution comprising 1.5 eq of Fmoc-L-Glu(NHCH₂CH₂STrt)-OH and 1.5 eq of HOAt was prepared, followed by ice bath, and then 1.5 eq of DIC was added to react for 10-15 minutes. Subsequently, the activated amino acid solution was added to the deprotected resin to react for 60-90 minutes, and then 0.5 eq of PyAOP and 1.0 eq of DIPEA were added to the reaction mixture to react for 60-90 minutes, and the coupling reaction was monitored by ninhydrin test, and the test result should be negative. After the coupling was completed, the Fmoc protective agent was removed by the method in step 2, and ninhydrin was used for sampling and testing.

### 4) For Amino Acid No. 1 (Fmoc-L-his(Trt)-OH)

5.0 eq of Fmoc-L-His(Trt)-OH, 4.75 eq of TDBTU and DMF were added into a reactor containing the deprotected resin, then 10 eq of DIPEA was added to react at 20-30° C. for 120-180 minutes. The coupling reaction was monitored by ninhydrin test. If the reaction result was negative, the reaction was completed. After the coupling of Fmoc-L-His(Trt)-OH, the N-terminal Fmoc protection was removed. After the ninhydrin test was positive, methanol washing was carried out for four times.

### Step 4: Pyrolysis and Purification

After being washed, the polypeptide resin was dried under reduced pressure to constant weight, then a precooled lysis buffer was added thereto, and the mixture was stirred for 2.5-3.5 hours, to obtain a fully assembled polypeptide by cleavage from the resin, and the protective group of the amino acid side chain was removed.

The excess resin was removed by filtration, and the filtrate was added to precooled ether to obtain a precipitated product, which was then washed with ether and dried to obtain a crude product.

Luna C18(3) resin was used to purify the crude product in two stages, to obtain a final product. The purification conditions were shown in Table 1 below.

TABLE 1

| | Conditions | |
|---|---|---|
| Parameters | Primary purification | Secondary purification* |
| Mobile phase A | 0.1% trifluoroacetic acid | 0.5% acetic acid aqueous solution |
| | aqueous solution | |
| Mobile phase B | 0.1% trifluoroacetic acid: 80% | 0.5% acetic acid: 80% |
| | acetonitrile: 20% water | acetonitrile: 20% water |
| Chromatographic | Luna C18 (3) resin | Luna C18 (3) resub |
| column | | |
| Flow rate | 400 mL/min | 400 mL/min |
| Fraction | Main fraction: purity ≥90.0%, | Main fraction: purity ≥98.0%, |
| acceptance | single impurity <2.0% | impurity (RRT about 0.86) ≤0.3%, |
| standard | Recovered fraction: purity ≥80.0%, | single unspecified impurity <0.5% |
| | but it didn't meet the requirements | Recovered fraction: purity ≥90.0%, |
| | of main fraction. | but it didn't meet the requirements |
| | | of the main fraction. |

*Before elution with mobile phases A and B, the product was subjected to salt exchange with 32 g/L ammonium acetate in the secondary purification.

### Example 2: Synthesis of Polypeptide Conjugate I-1

Polypeptide conjugate I-1(PEG molecular weight is about 23 KDa) has the following structure:

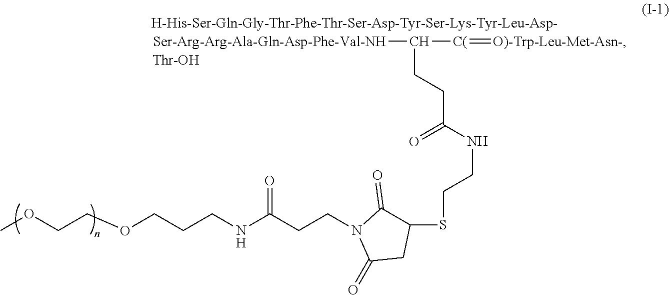

The synthesis steps of polypeptide conjugate I-1 were as follows:

### Step 1: PEGylation

A solution containing 70% buffer solution (Na₂HPO₄+NaH₂PO₄, pH 6.5) and 30% acetonitrile was prepared, to which 1.1 eq of α-[3-(3-maleimido-1-oxopropyl) amino] propyl-ω-methoxypolyethylene glycol-23 KDa was added, and stirred for 5-10 minutes. Then glucagon mutant Q-1 was added and stirred at room temperature for 1-2 hours under nitrogen protection. The end point of the reaction was determined by HPLC, and the residual glucagon mutant Q-1 did not exceed 0.5%.

### Step 2: Purification

After the reaction was completed, the reaction solution was diluted with water, to adjust the pH to 4.0, followed by chromatography with an ion exchange column. The purification parameters were as follows. After ion exchange purification, desalination-concentration-ultrafiltration processes were carried out, followed by freeze-drying, to obtain a polypeptide conjugate I-1 product.

TABLE 2

| Parameters | Conditions |
|---|---|
| Mobile phase A | An aqueous solution of 3.6 mmol/L acetic acid and 16.4 |
| | mmol/L sodium acetate was adjusted to pH 4 with acetic |
| | acid |
| Mobile phase B | An aqueous solution of 3.6 mmol/L acetic acid, 16.4 |
| | mmol/L sodium acetate and 1 mol/L sodium chloride was |
| | adjusted to pH 4 with sodium hydroxide |
| Chromatographic | Ion exchange resin |
| column | |
| Flow rate | 200 mL/min, or the maximum flow rate within the column |
| | pressure limit |
| Fraction acceptance | Main fraction: purity ≥97.5%, single impurity <1.0%. |
| standard | Recovered fraction: purity ≥90.0%, but it didn't meet the |
| | requirements of the main fraction. |

### Experimental Example 1: Evaluation of the Effect of Raising Blood Glucose In Vivo

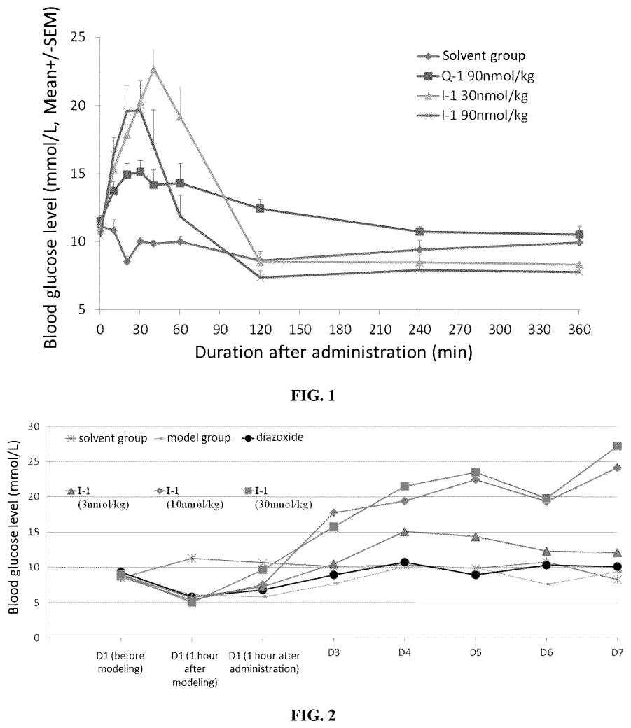

20 C57BL/6j male mice aged 4-5 weeks were randomly divided into 4 groups according to their body weight, with 5 mice in each group. All animals were injected with normal saline (0.2 ml/mouse) twice a day in the morning and afternoon during the one-week quarantine period in advance, and the animal feed was removed 1-2 hours before the experiment (fasting, with supply of water). The administration conditions for the respective groups were as follows: a solvent group (buffer solution), a low-dose group of polypeptide conjugate I-1 (30 nmol/kg), a high-dose group of polypeptide conjugate I-1 (90 nmol/kg) and a high-dose group of glucagon mutant Q-1 (90 nmol/kg). The blood glucose of each animal was measured before administration and at 10 minutes, 20 minutes, 30 minutes, 40 minutes, 60 minutes, 2 hours, 4 hours and 6 hours after administration. The test results are shown in FIG. 1.

A shown by the results, both polypeptide conjugate I-1 and glucagon mutant Q-1 were capable of significantly raising blood glucose, and the PEGylated polypeptide conjugate I-1 had a more obvious effect of acutely raising blood glucose. The low dose (30 nmol/kg) polypeptide conjugate I-1 achieved a rate and peak value of raising blood glucose much higher than those of the high dose (90 nmol/kg) glucagon mutant Q-1.

### Experimental Example 2: Evaluation of Glucose-Raising Effect of Multiple Doses on Glimepiride Hypoglycemia Mouse Model

Glimepiride (CAS: 93479-97-1) is a sulfonylurea hypoglycemic agent, and it has been reported that oral administration of glimepiride may cause hypoglycemia in experimental animals^{[9]}. Herein, a hypoglycemia mouse model was established by taking glimepiride orally according to the dosage in the literature, and 20 C57BL/6j male mice aged 4-5 weeks were randomly divided into 6 groups according to their weight, with 5 mice in each group. The model group, positive control group and respective compound (I-1) groups were given glimepiride at a dose of 10 mg/kg by gavage, once every morning and afternoon for 7 days, while the solvent group was given 0.5% CMC-NA solution with the same volume. 1 hour after the intragastric administration of glimepiride every morning, compound (I-1) was injected subcutaneously, or diazoxide, a positive drug, was administrated by gavage for 6 days, and the administration of the respective groups was as follows: the positive control group (diazoxide, 20 mg/kg), the model group (buffer solution), low-dose group of polypeptide conjugate I-1 (3 nmol/kg), middle-dose group of polypeptide conjugate I-1 (10 nmol/kg), and high-dose group of polypeptide conjugate I-1 (30 nmol/kg). Blood samples were taken from the tail tips of the animals to measure blood glucose levels before the first day of modeling, 1 hour after the first day of modeling, and 1 hour after the first and 3^{rd} to 7^{th} days of administration, respectively. The test results are shown in FIG. 2.

As showed by the experimental results, low-frequency injections of medium or high-dose polypeptide conjugate I-1 were capable of reversing the hypoglycemia of model mice for a long time.

### Experimental Example 3 Evaluation of Glucose-Raising Effect of Multiple Doses on Hypoglycemia Rat Model with Hyperinsulinism

40 male SD rats weighing 220 g to 280 g were divided into 5 groups, with 8 rats in each group, which were sham-operation group, model group, low-dose group of polypeptide conjugate I-1, middle-dose group of polypeptide conjugate I-1 and high-dose group of polypeptide conjugate I-1. On the first day of the experiment, all the animals were anesthetized with isoflurane, then a small incision was made on the back of the rats near the ilium, and a sustained-release pump was implanted in the back of rats in the model group and the administration groups, and then the wound was sutured. After the operation, each rat was given 0.2 ml meloxicam for analgesia for 3 consecutive days. The sustained-release pump released insulin at a rate of 2.5 ul/hr to cause hypoglycemia (a total dose of 80 nmol/kg). The sham-operated animals were also cut and sutured, but no sustained-release pump was implanted.

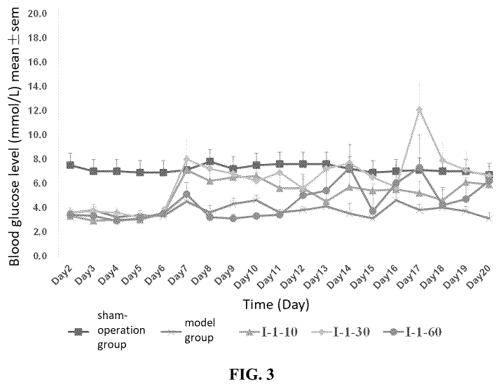

On the 2^{nd} to 6^{th} days of the experiment, the random blood glucose of animals in each group was measured at half past nine every morning. From the 7^{th} day of the experiment, the administration group was given subcutaneous injection once a day, wherein, low-dose group of polypeptide conjugate I-1 (10 nmol/kg, I-1-10), middle-dose group of polypeptide conjugate I-1 (30 nmol/kg, I-1-30), high-dose group of polypeptide conjugate I-1 (60 nmol/kg, I-1-60), and the sham-operation group and the model group were injected with the same volume of buffer solution. The blood glucose of each experimental animal was measured 4 hours after each administration. The results are shown in FIG. 3.

The results showed that long-term and low-frequency injections of polypeptide conjugate I-1 were capable of effectively treating hyperinsulinism, and in particular, the middle-dose of polypeptide conjugate I-1 was capable of restoring the blood glucose level of patients with hyperinsulinism to a normal level.

### CITED DOCUMENTS

[8] Bailon, Bioconjugate Chem, 2001, 12:195-202; Bowen, Experimental Hematology, 1999, 27:425-432: Bailon, Expert Opin Deliv, 2009, 6:1-16.
[9] Wu, Antidiabetics, 2009, 59(11):550-556: Ladriere, European Journal of Pharmacology, 1997, 335:227-234; Murthy, Ther Adv Eedocrinol, 2013, 4(5): 133-138.

All technical features disclosed in the present specification may be combined in any combination. Each feature disclosed in the present specification may also be replaced by other features with the same, equal or similar function. Therefore, unless otherwise specified, each feature disclosed is only an example of a series of equal or similar features.

In addition, based on the above description of the present disclosure, persons skilled in the art are able to easily understand the main features of the present disclosure. Without departing from the spirit and scope of the present disclosure, many modifications may be made to adapt to various use purposes and conditions, so such modifications are also intended to fall within the scope of the appended claims.

1-12. (canceled)

13. A method for treating a disease related to glucose metabolism, wherein the method comprises administering to a subject a therapeutically effective amount of a polypeptide conjugate formed by conjugation of a glucagon mutant with one or more polyethylene glycols or a composition comprising the polypeptide conjugate;
and the glucagon mutant has substitution, addition or modification of amino acid at one or more positions of a sequence shown in SEQ ID NO: 1, so that a side chain of at least one amino acid contains a sulfhydryl group, and the polyethylene glycol is conjugated to the mutant through the sulfhydryl group.

14. The method according to claim 13, wherein the substitution, addition or modification of amino acid includes at least one of the following (a) and (b):
(a) the amino acid at one or more positions of the sequence shown in SEQ ID NO: 1 being replaced with cysteine,
(b) one or more cysteines being added at one or more positions of the sequence shown in SEQ ID NO: 1.

15. The method according to claim 13, wherein the substitution, addition or modification of amino acid is that a sulfhydryl-containing group is connected to the side chain of the amino acid at one or more positions of the sequence shown in SEQ ID NO: 1.

16. The method according to claim 13, wherein the substitution, addition or modification of amino acid is at least one of the following (c) to (g):
(c) a sulfhydryl-containing group being connected to the side chain of glutamine at position 3 of the sequence shown in SEQ ID NO: 1,
(d) a sulfhydryl-containing group being connected to the side chain of lysine at position 12 of the sequence shown in SEQ ID NO: 1,
(e) a sulfhydryl-containing group being connected to the side chain of glutamine at position 20 of the sequence shown in SEQ ID NO: 1,
(f) a sulfhydryl-containing group being connected to the side chain of glutamine at position 24 of the sequence shown in SEQ ID NO: 1,
(g) a sulfhydryl-containing group being connected to the side chain of asparagine at position 28 of the sequence shown in SEQ ID NO: 1.

17. The method according to claim 13, wherein the substitution, addition or modification of amino acid is that a sulfhydryl-containing group is connected to the side chain of glutamine at position 24 of the sequence shown in SEQ ID NO: 1.

18. The method according to claim 15, wherein the sulfhydryl-containing group is —C(═O)—Z₁—SH or —Z₁—SH, Z₁ is an unsubstituted or amino-substituted C₁₋₄ alkyl, the non-sulfhydryl side of the sulfhydryl-containing group is connected with the amino group in the side chain of the amino acid.

19. The method according to claim 18, wherein the sulfhydryl-containing group is —C(═O)—CH(NH₂)—CH₂—SH, —CH₂—SH, —CH₂—CH₂—SH or —CH₂—CH₂—CH₂—SH.

20. The method according to claim 18, wherein the sulfhydryl-containing group is —CH₂—CH₂—SH.

21. The method according to claim 13, wherein the glucagon mutant has a sequence shown in any one of the following sequences:
(SEQ ID NO: 2)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln
(-CH₂—CH₂—SH)-Trp-Leu-Met-Asn-Thr-OH,
(SEQ ID NO: 3)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln
(-CH_{2—}CH_{2—}SH)-Trp-Leu-Met-Asn-Thr-NH₂,
(SEQ ID NO: 4)
H-His-Ser-Gln-Gly-Thr-Phe-Thr-Ser-Asp-Tyr-Ser-Lys
(-C(═O)—CH(NH₂)-CH₂—SH)-Tyr-Leu-Asp-Ser-Arg-Arg-
Ala-Gln-Asp-Phe-Val-Gln-Trp-Leu-Met-Asn-Thr-OH,
(SEQ ID NO: 5)
H-His-Ser-Gln-Gly-Thr-Phe-Thr-Ser-Asp-Tyr-Ser-Lys
(-C(—O)—CH(NH₂)-CH₂—SH)-Tyr-Leu-Asp-Ser-Arg-Arg-
Ala-Gln-Asp-Phe-Val-Gln-Trp-Leu-Met-Asn-Thr- NH₂,
(SEQ ID NO: 6)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Cys-
Trp-Leu-Met-Asn-Thr-OH,
(SEQ ID NO: 7)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Cys-
Trp-Leu-Met-Asn-Thr- NH₂,
(SEQ ID NO: 8)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Cys-OH,
(SEQ ID NO: 9)
H-His-Ser-Gln-Gly-Thr-Phe-Thr-Ser-Asp-Tyr-Ser-Lys-
Tyr-Leu-Asp-Ser-Arg-Arg-Ala-Gln-Asp-Phe-Val-Gln-
Trp-Leu-Met-Asn-Thr-Cys-NH₂.

22. The method according to claim 13, wherein the polyethylene glycol has a molecular weight of 1 KDa to 60 KDa, 5 KDa to 50 KDa, 10 KDa to 40 KDa, 15 KDa to 30 KDa, or 21 KDa to 29 KDa.

23. The method according to claim 13, wherein the polypeptide conjugate has a structure shown in formula (I-1) or (I-1′):

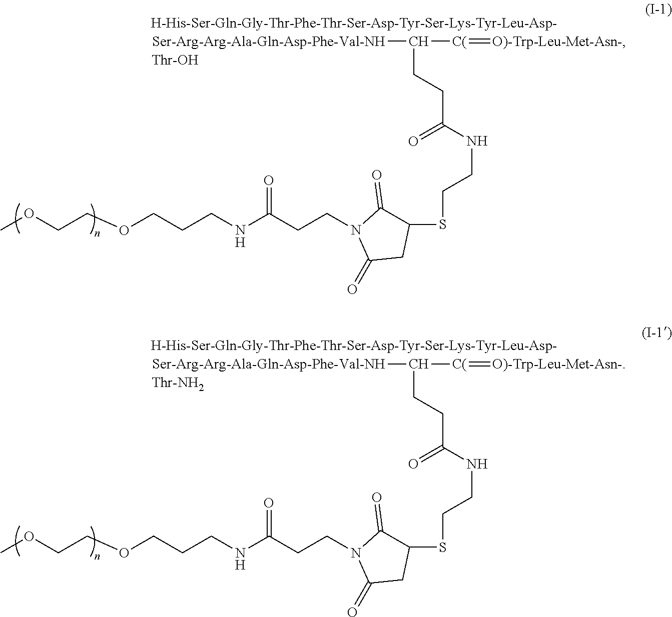

24. The method according to claim 13, wherein the disease related to glucose metabolism is hypoglycemia, which is any one selected from congenital hyperinsulinemic hypoglycemia, diabetic hypoglycemia, non-diabetic hypoglycemia, reactive hypoglycemia, fasting hypoglycemia, gestational hypoglycemia, drug-induced hypoglycemia, surgery-induced hypoglycemia and tumor-induced hypoglycemia.

25. The method according to claim 13, wherein the disease related to glucose metabolism is congenital hyperinsulinism.

26. The method according to claim 21, wherein the disease related to glucose metabolism is hypoglycemia, which is any one selected from congenital hyperinsulinemic hypoglycemia, diabetic hypoglycemia, non-diabetic hypoglycemia, reactive hypoglycemia, fasting hypoglycemia, gestational hypoglycemia, drug-induced hypoglycemia, surgery-induced hypoglycemia and tumor-induced hypoglycemia.

27. The method according to claim 23, wherein the disease related to glucose metabolism is hypoglycemia, which is any one selected from congenital hyperinsulinemic hypoglycemia, diabetic hypoglycemia, non-diabetic hypoglycemia, reactive hypoglycemia, fasting hypoglycemia, gestational hypoglycemia, drug-induced hypoglycemia, surgery-induced hypoglycemia and tumor-induced hypoglycemia.

28. The method according to claim 21, wherein the disease related to glucose metabolism is congenital hyperinsulinism.

29. The method according to claim 23, wherein the disease related to glucose metabolism is congenital hyperinsulinism.


SEQUENCE LISTING (9 sequences)

SEQ ID NO: 1 - protein, 29 aa, synthetic construct
  REGION 1..29: note = glucagon sequence
HSQGTFTSDYSKYLDSRRAQDFVQWLMNT

SEQ ID NO: 2 - protein, 29 aa, synthetic construct
  MOD_RES 24: note = Attached to 2-mercaptoethyl
  REGION 1..29: note = glucagon mutant sequence
HSQGTFTSDYSKYLDSRRAQDFVQWLMNT

SEQ ID NO: 3 - protein, 29 aa, synthetic construct
  MOD_RES 24: note = Attached to 2-mercaptoethyl
  MOD_RES 29: note = AMIDATION - -NH2
  REGION 1..29: note = glucagon mutant sequence
HSQGTFTSDYSKYLDSRRAQDFVQWLMNT

SEQ ID NO: 4 - protein, 29 aa, synthetic construct
  MOD_RES 12: note = Attached to 2-amino-3-mercapto-1-oxopropyl
  REGION 1..29: note = glucagon mutant sequence
HSQGTFTSDYSKYLDSRRAQDFVQWLMNT

SEQ ID NO: 5 - protein, 29 aa, synthetic construct
  MOD_RES 12: note = Attached to 2-amino-3-mercapto-1-oxopropyl
  MOD_RES 29: note = AMIDATION - -NH2
  REGION 1..29: note = glucagon mutant sequence
HSQGTFTSDYSKYLDSRRAQDFVQWLMNT

SEQ ID NO: 6 - protein, 29 aa, synthetic construct
  REGION 1..29: note = glucagon mutant sequence
HSQGTFTSDYSKYLDSRRAQDFVCWLMNT

SEQ ID NO: 7 - protein, 29 aa, synthetic construct
  MOD_RES 29: note = AMIDATION - -NH2
  REGION 1..29: note = glucagon mutant sequence
HSQGTFTSDYSKYLDSRRAQDFVCWLMNT

SEQ ID NO: 8 - protein, 30 aa, synthetic construct
  REGION 1..30: note = glucagon mutant sequence
HSQGTFTSDYSKYLDSRRAQDFVQWLMNTC

SEQ ID NO: 9 - protein, 30 aa, synthetic construct; identical to SEQ ID NO: 8
  MOD_RES 30: note = AMIDATION - -NH2
  REGION 1..30: note = glucagon mutant sequence